SEQUENCE LISTING (23 sequences)

SEQ ID NO: 1 - DNA, 2016 bp, Homo sapiens
gccagctggagcgttttcggggctgtaaagggagaatggcgcatgcgcatattcagggcggaaggcgcgctaagagcagatttgtggtctgcattatgtctggagccagaagtaagcttgctcttttcctctgcggctgttacgtggttgccctgggagcccacaccggggaggagagcgtggctgaccatcacgaggctgaatattatgtggctgccgtgtatgagcatccatccatcctgagtctgaaccctctggctctcatcagccgccaagaggccttggagctcatgaaccagaaccttgacatctatgaacagcaagtgatgactgcagcccaaaaggatgtacagattatagtgtttccagaagatggcattcatggattcaactttacaagaacatccatttatccatttttggacttcatgccgtctccccaggtggtcaggtggaacccatgcctggagcctcaccgcttcaatgacacagaggtgctccagcgcctgagttgtatggccatcaggggagatatgttcttggtggccaatcttgggacaaaggagccttgtcatagcagtgacccaaggtgcccaaaagatgggagataccagttcaacacaaatgtcgtgttcagcaataatggaacccttgttgaccgctaccgtaaacacaacctctactttgaggcagcattcgatgttcctcttaaagtggatctcatcacctttgataccccctttgctggcaggtttggcatcttcacatgctttgatatattgttctttgaccctgccatcagagtcctcagagactacaaggtgaagcatgttgtgtacccaactgcctggatgaaccagctcccactcttggcagcaattgagattcagaaagcttttgctgttgcctttggcatcaacgttctggcagctaatgtccaccacccagttctggggatgacaggaagtggcatacacacccctctggagtccttttggtaccatgacatggaaaatcccaaaagtcaccttataattgcccaggtggccaaaaatccagtgggtctcattggtgcagagaatgcaacaggtgaaacggacccatcccatagtaagtttttaaaaattttgtcaggcgatccgtactgtgagaaggatgctcaggaagtccactgtgatgaggccaccaagtggaacgtgaatgctcctcccacatttcactctgagatgatgtatgacaatttcaccctggtccctgtctggggaaaggaaggctatctccacgtctgttccaatggcctctgctgttatttactttacgagaggcccaccttatccaaagagctgtatgccctgggggtctttgatgggcttcacacagtacatggcacttactacatccaagtgtgtgccctggtcaggtgtgggggtcttggcttcgacacctgcggacaggaaatcacagaggccacggggatatttgagtttcacctgtggggcaacttcagtacttcctatatctttcctttgtttctgacctcagggatgaccctagaagtccctgaccagcttggctgggagaatgaccactatttcctgaggaaaagtaggctgtcctctgggctggtgacggcggctctctatgggcgcttgtatgagagggactaggaaaagtgtgtggtctgtggggcggactctggccatcatgttgacagccttgcacttccacaggctacaagccctgggaccatctttctgccttaagggcaggagcccacttctgtggcaccagattccaccctgggaactgtggaaaaagtaggagaggcagattccctcagtgtcttcctcttaaacctcaatcatcgagacattagggggtattttctgttcacatttatctttttcaagccacatcttcctctaacaaatctctcagtatgcgattggtctcaagctaaaacaaaaataaatgtcagtttatattttacacatccaaaaaaaaaaaaaaaaaaaa

SEQ ID NO: 2 - DNA, 1000 bp, Homo sapiens
cttccctccctcccgggcgctaaaaggaaaaccccccgacccccatcgcccatttctactcgtctccaagacaacatcgcggtccccgccagcttccgtaggagcctttcattccaggaaggtccatcgtacttgcgttttcagggcctgagcgatgactttagcaccagacacctgctcctcgctgcgctctgcgaagttactgtccggcatcttccaccgaaaagctctaagcactcacgcagccggcaaacaagcggaatcatccagcaaggcaaacgcgaagtcggcagcacgccacctctggtactgcacctctgacggacaggagggcaaccaactgccttaaacaacgggaaggaagaggcggtctaaattcgtccacttcccgggagaggtgagaatgtaaacacgcgcattctccaatcagaactgcgctctcttctcggctcctccattcgcgcgccagaatgccagagggaggcgggactagcaggagattgctgcctatgcaaagcaggtaagaagccgaactctgaggcctctcgccattgtctccgagtcggccagctggagcgttttcggggctgtaaagggagaatggcgcatgcgcatattcagggcggaaggcgcgctaagagcaggtacggagggggcgtggtgcggcgcggagggggtgtggtaagggcgtgcggtccagaccccgccccgggcgcccagttggacttggggagggctgcgcaaaggctgccgggagctgggaagcccggcgcgcgtcgtttgctggggctgtttgtgcgttgctgctgtgctaccgcgttgcgttttctaggcatttacttacacgctttgtggtttacgctctcataaccttgtggttttatagtccttaaattattgtagcgcacgttacttaaatccagaagcagatgtgtaccccagcaagagataaaatgacgctcagagtcagtagatccagaccgtgcctgagatcctgaat

SEQ ID NO: 3 - DNA, 12990 bp, Homo sapiens
tctcactggctgctcttatgatccagaatggaagaggatgaggacaaatgcagggggatgttaggagaccactaagcaggtccctgtcatttctctctctgtgattccttttgctgccactttctccttgtccccttggctccagccccctgctgtccttgctgtttcttgcacccgcccgtgaggcatgctcctgcctcaggctctctgcctgccgtgctctcacctcgcagacccacgtgatttcctcccgaacccccttcaggcctcggtccaaacatcaccctctcatcgaggtcttccttgaccacactgcttaaaattgtccccctcgcctcaccttaccaccttcactgcctcatttctcctttgtgcttaatcaccatctcacacagttatccagagcacgagccccacgaggaagggtctgtcttgtccactgttggacccctgacacctaacccctggggctggcccacagttgggaattgcatggctgtctgaagccagcaccttcctggttctgctgttccctggaagtggggttcagtacaccccacgagccaaggccctcatctcagagggcgtgcacatggttgcctcaattgtgctttcacactggacccttcctgcagtttactctcctatgtcagatgcccttcaatgaaagcaagtacattgccaccttgtcacacctctagttaccattttctttatggtccaggtcctgaccagctctaaagatgggtcagctgctgtatttccaagaaccccatgacttccagggccccttgttcccaggaaggccccgcaccccagtcgtcccgtgcataacctcacgggccagcacctggtagctgctggaaggcttctgggggatgcctgggcccctccagccctacctctagtcctgccactttacaactgagcacctcccgctgctgcctgctgaacccttcagagtccttgccacaggccctatttactttttcctgagaaggtatgtgtgatgccaaagagagaaaagcagcatttgctaatttgggtaaaatgtttctttgggaagggcaaattgatgtacagttgtccctcagtatctgttgggactggttccaggaccccgtggataccgaaaccctcagatgctcaagtcccttatatcaaatggtgtagaatttacatgtaacctaaacacttcctcctgtacactttaaatcatctctagatcccttacactacctaatacaaactaaatgctatataaataattgttatactgtattttttaaattgctattattttttattgttgtgctattatttttattgggctccctcctccccctcccaccccagtaatttcaatccaaggttggtggaatccatggatgcagaaacaatggatactgagggccggctgactagacctatttttattgtaattactcattctcttctacctcattaagcttggttctttcaagcatgaatacctggttgaatctgcataacctcttattatcgacctacctttgttcacacacaaatgattgccacttagagctctcctaccgggctccttttgtaaaataaattttatcttctccagatagaaagaaatgtgataccttgcggattttgcggtttccttgttttgctggattgcaagtcctttagacataaacaaatgttctgtaccccgacatcggtggtacccagctctcagttgagaaagagatgtaatgtgaatgccactcttggccccaggcactcctcacttgcccccactggggactggttcataccctcctctcctggcctcagtttccatacctcttagtgaggcctttgcgttatacttcagaaatattgtcagtatgactttgaagatgaaagtttgcccccaaaatcactctctgttatcattgtgaaaccagagatggaatggaaaaatgggtttctgagacattttaaattattccttgcttgtctttagaggcaaaattcagataagaaagcttatcaattatacttttgtttctactcaaaaactcatgactgctcactcaaagactccttgttcttatcttaaaacattgtttacagtgtcccagatgaatggcaacaaatcctggggtttggtgtttggatggtacatttccctggggaaagaaataacagtttgagatggaacggggttggggtgggagaatacttctcattctgaggaatatttaattttgccaagatgagcatttctagttcttagtctgttgacgaaaagagctatggtttgtttctggaacttttgataaaaaataaagaaatttgtagcctggggagtttggttttaaaatgcaaacacaggagttatgagttgagacttggacagggtgtcattttctttttaaagggcagcaatatgattctttgatttgtctttgttatcttgacttttaatccggattcctgggcagttgttcagccccaggacatctccatgggcaggtggcctggccttggcacactacccagtaaatctctgcctgagaggacgctttagctgggaggccaggctgatttttaaaggcagaattggactatttactctaaaacagtaatgcacactgtttagaaagaaacattcctattctgggaggaaggaggagacacacagaagtatcatttatttctagtcttttctggtagaagctatgaagctgagtttactctctggaaatttgtagtttattttctagaaaattgcattttatcactgcaaaaaggattttatttccaaatgagtaggcttttgagcaagagttttggagtcacagagatggggttaagaaagtgataatgtgcaatggcgattctcaagttcaaggagaaaaaataacatgcttttattgggatactttgcttgtctataaaagaaagtagctattggcatttatgtagaagtcagcagtttcttggcaccaaataaataattttgtgctgaataaagggagagttatccatagtatttattactaaccaaagaaatgcagggagaattgtaattcattaggttttgatggccaggaaagccaagctgtgttattagggtcatgacaatcacagacattacggatggctgacctgtagtatggatagagggcagagggtagagtgtgaaatatatcacagaattatgtcaaataatctggatagttactactgcttaaaatctaagtgcacagctagaaaagtgggtagtgacgcactacagtcttgctgaacactgggtaagaaaatcatagcaaacgttgagtctgttttggaaatgttctaaaaccagactattaacacagtgagccattttaaatgtggcttgctacgtgtttggagagaaacacatactcttttattaggaacatgaaacaaactctttgagccgcagtatcactgcgagtgagtttaattgctgggattaataaatcacagctgcaaacgttaaattcttggcaggattctttattcagctgttttccccttgccccattacattccagatttgtggtctgcattatgtctggagccagaagtaagcttgctcttttcctctgcggctgttacgtggttgccctgggagcccacaccggggaggagagcgtggctgaccatcacgaggctgaatattatgtggctgccgtgtatgagcatccatccatcctgagtctgaaccctctggctctcatcagccgccaagaggccttggagctcatgaaccagaaccttgacatctatgaacagcaagtgatgactgcagcccaaaaggcaagaatgctcctcggaacctgagtttctctcatacagagcagattgctctttaccccttgatcagtggttgggtaatcccaggcttcctaccaccctctgaaaaagcatccaggtagttaacctgagttgagttagtcagttgaattaggagccttacccctcagagagtggtccgtggaccggcatcccctgggagcttgttagaaatacaaaatcttgggcggcaccccagacctactgaatcagaatgtgcattgcagcaggatccccaggtgatgctttcacatggcaagtatgagaagcccaggactagatccccagttctcaagtgtggttgtacataagaatcacgaggtaagtggtaaacactatggctgcccgggtcctggagagtccgttgtaattggtgtggaaggggtgtggactggcactgggattgttttaaggctccccagtgcagtctaatgtgcagaaaaaatttgaaagatgactgggcgtgatgacctctctgagtcattcgaagcttcactgaagtagtaagcatctgcaagaatgccgtttgctcccttcagactgtttgaggctcgtttccggtctctatgtcggactacgatcagtctgagaccttcgcccagatagaactgaccccaaactgacaaagggaaggtcagtgccagcctttgtgaaggcttcctggttggcctgaatttcctgctcccttcaggaagggtggggacaaaggagaggcccccctgggggcaaagagggaaatatcagaggttgcctaagaaaatgccctgctggaaaacacaaacccgaagggaagtttgggctgtaactctggtggcagggtgaccaagcgcagctgcttgaggaagccctgctgtgcctcaacaggatgtaaactcattgtgagcaacactttcctgctctctgtgaacttaaagggcagaaccagcaggtcctgccccaaacagtccctgccttagagcagggtggtcgggatggcctggacagccacagcaattaaaaaattgcaacattttaaaattttagtctataatatatatacaaaggctatgtgtatggggtgggggggtgtttgggggcagggggtgtgtatgtgtgtataacatgatgttgaaagggaacttgaagacttggtccagcttcttttttttcaaccaagaccaacttttgcaagggtgacacttttctttagtcccaacctgacatacggtttctccttgaacaccttcagtggctcagactcacaggtccgtttgttccaatggtgggaacttctgaacaggtcttcctttcaatgagcagcagtcagcctccccgtaactgccaccacgattctatcgtcagagctaaagggagcaggaccgtgtcccttatcacggcatgccatcttctccacctttgaggacagctgtcatgatccccctggcatctgtcccccaggctgtatcctcagtcccttccacagttccttaggagactcagtttccaaaccttctactgaagacttccatgttttctctgtgctcagaactgtatgcagctatcccgattctgtctaataagggcagggtagagaactctcacctgtcgcattctagatgttgtccccagaaaactgctggcagccacatgtctcattatgggtgtataaggcacttgctgtcaactaaaacaccttttcacatgagcagacacacatgctgccattgccatcctgtacttataaattataaaggtgattgatttaagctgagggcaagacttcacatttatgctgttaaatttcatcattccagcctgttgggctatttagggatctttactgacatcccaagtatcagttacctttacgtcattcacacatatgatacacacctcatttatgtctatgctgaagtcagtgtaaaaaaaccccaggctgtgccctcagacctcctgatgacactgatctcctagagggcaggcattctcttgatagagatgtttgcctgcatggcactgagtccagcacctgaaatgtcatctgcctcttgcttccctcccctatccaccggaccattctgagacatttggcaaatgacacactgaaacccagactgtggctgtagaattctcctgcattcacctttcaataatctgcccccagaggaaacacttaacacggttttgttgaaaccacgccagctgcacagcatcactccgtctctatttgttttccaggggccaggattaagctgttgatatgatcacttttagaatttacagatatctcagctcccatacgtggttatatgttttttatttgtttgttttccagcagcacttttattttccttacacgatgacatgttgctggggcctattgttctcacataacagtagaaaaccaaaatttgttgtcatctcttcaaagaatcgagaattgcatacagaaaaaccttacataaattaaaaggatgaatacatttacaggtgtaaatgcaaaccactttcaactcagacaagtaacagcccatggtgttctggcagaaaacatcagctaagaaaggaaactgggtcctaagtcttggactttccaacccttacagaccggcagaacagaaacaactggttcaggagcccttgccagcctccagagaaatcccagaacacgcagccctgacgtattaataccctgcacagatcagagactgctggccacgcagactcaccaagccacagacttgtcttccacaagcactttcttatcttagccacaaagtgaccaagccacatgtactaagggttgaaatcaaagatatgtacagggtattaagcaaatctggttatatgttttaaaacaacttctaagacaaattgatggcaagtttgtgtgaaagttttatatcaaagttgttataagaggttcctgagcaaaccaattgaaatacagtcatgcattgcttaatgacagggatatgttctgaaaggatgcatcattaggccattgtgtcattgtgcatgcatcatagcatgtacttacacaaacctacatggtacggcctactatgcgcctaggctatatggtatggcccattgttcctaggctataaacctttacagcatattactgtactgaacactgtaggcagttgtaacaagtggtaagcatttgtatatgtaaacatagaaaaggtacaataaaaattcagtattataatcttatgggaccaccatcacatatgtggtctgtcattgaccaaaatgtcatcatgcagtgcatgactatatttctgtctcagtaggggcattcataggggaaaaacggagtctagtttcaagatgattaggctgggcagtcacttgggattgtaaccttcattcctcagaaggaaggggttcttgatctcattgagatctaccagaaaattgctgaagccatttatcaagaatgcaacttacttcctagataggattactcatcacatcagacccaaaattttgcccagctcaggtttggttcctctcctcattcctggttgataataatctagtatgtatacataatttaaatgttattctccatgaaaaaccaaagttttgtttttaataaagaaaaatgtctatccaaatataattttcaaaaatctgaaaagatgactcatacaaatatagaatgaataaagcttttatttaattcattaattaaggaaccagtaagatggtaaagctggttcaaaggaaaattcaaggaatggaaatgtgtatatcagtcagtccagtgattgttgaaatgaatttcctaatagatgcaaaactgggtaatgtcctatagggcaaaacattgtaatctttgaggtgatcttttaaatagcaaagtcaaacggtggtacattctccagctaattaaagaataattgagtgagcctattaaacagtaccctagtataatttggaaaggctgcatctccatcttgccttatttttaggtttgagataatttttctttacatggtcattgctaagtgtgcaatgagatgatactgtactggaaggaacatacattggtatagtatttctggaaagcagtttggcagtgtgtgttaagaacttaaaagtttaatttttaggccaggtgctgtggctcatgcctgtaatcccagcattttgggggtccaaagcgggcggatcacttgaggtcaggagtttgagaccagcctgatggtgaaaccccatctccactaaaaatacaaaatttagccaggtgtggtggcgcatgtctgtaatcccagctactcaggaggctgaggcacgagaattacttgaacccaggaggcggagattgcagtgagccgagatcacaacactgcactccagcctgggcgacagaccaagactctctctcaaaaaacaaaacaaaaattaaaactctaatttttataccctttgatccagtaatttcacttgtaagactttattccaaagaaataatcaaaagatgcaatcaaagatttgtgtgaagtgtataattatgcaataagtgttttgagcacactatgcagatggtcaccacagttttctttttattacaaaaagttgggaacacttcaaattccaataatagaggataaattatggcgtcctcttaaatatgatgtggccccattacaaatggatttttgaaagttttttttttttccttttttttttgtggtggagtttcactttgtcacccaggctggagtgcaatggtgcgatctcagctcaccgcaacctctgcctcccgggttccagtgattctccagcctcagcctcctgagtagctgggattgcaggtgcccgctaccatgcctggctaatttttgtatttttagtagagacggggtttcatcatgttgggcaggctggtcttgaactcctgagctcaggtgatctgcccacctgggcctcctgaagtgctgggattacaggcgtgaactgccatgcttggccgtattttttaaagttcttaatgagggaagtcaagatgtaaaaccatatatttattattatctccattatatacacacatacatgtatacagagagaaaaagtaatgaaaataaccaaaatattaacaataagtatctgtgttatagaattatgattgttttttcccgttttccaaattttctacagtaaaacttttgaagcttttataaccaggaaaaaaatttaaaagtttgcaatgcattccagaaataagtgtctcaaactttgctaatttgaattgttcatgccttctctgcctgccttctccaccttcctccctggggctggtgttcccggcttgacattttaaaccctgtaagtggagagcagtggaagaatgatgccccagccctgagagctgagggcggccctgtttgtattttcttaggttgctgtagatgtcacagggagttccgggccatcacagccagggaacacaggatgttgccaggtgtgggaaaaggcctttagggtggtcagagtcccgaagggagcctcctaattcccagttggggaatggagatttcaagcgagttcttgtttccaggctgagatgagcacacttgcctcttacccactggcccagtggatcctaaccttggctacaaatgagaatcacccgggggacctttaaacaaacactgttgccactatcccacccacagtcaatcaaatcagactttgtaggggtggtcccggcatcagtggtttttcagaagttcctcaactgatttaaatgcacaatggaagttgacaaccaccagactgaagataccacgtgtgttaatgggcccaatgtattcaaggcccagtagttggccccatctcccctggtatcctaagaactctaaatcctttctagctattcgcttgtcaaactcctgagcttactttcaatggagcttacacattccctccttccctcacatgaccccaggcacagttaatggttgttcctagaggactttgtctttgttccttggggatcaggtggagtgagacagtatccccaagactaagatctctgaggagagtaaagacaccatctctgtgcctctggttcctgctacagagtaacttcctgatggttgccaaaagaatgaacagaagaatgaatgaatgcagcggttcttcctgccatctgataacagactattctttgatgttttcattttcaggatgtacagattatagtgtttccagaagatggcattcatggattcaactttacaagaacatccatttatccatttttggacttcatgccgtctccccaggtggtcaggtggaacccatgcctggagcctcaccgcttcaatgacacagaggtgattcctgcctttttcctcagtaggctgagggtacacagaggtgatctaagtcagggaccagaagctgtgacatgttaactaagattgataggagaccttaacatccccaaaatccaacccaaactcccaaagatccatgtgccacatgttcattccattaaagaatgtctgacgttacaaggcagttattcatctatggatctttccatttattaattacacaataaatacaggaatgtatacttaaaccaaaccaaaagtaaaaaaagaaaagttcatcttcaccacagcctgcacctcatcccatgcccttgcttagagaaactgccatcaacaatttgatgtgcattcagttgtattcttttctatgcatttcatagttattgacatcctctttttttttttttttttgagatggagtcttactctgccacccaggctggagcgcagtggcgcgatctcggctcactgcaagctccgccttctgggttcacgccattctcctgcctcagcctcccgagtagctgggactacaggcatccaccaccacgcccggctaattttttgtatttttagttgagatggggtttcaccgtgttagccagggtggtctcaatctcctgacctcatgagccacccgcctcagcctcccacagtgctgggattacaggcaaaaacctcatttatttacacctttttttcctctaggtgctccagcgcctgagttgtatggccatcaggggagatatgttcttggtggccaatcttgggacaaaggagccttgtcatagcagtgacccaaggtgcccaaaagatgggagataccagttcaacacaaatgtcgtgttcagcaataatggaacccttgttgaccgctaccgtaaacacaacctctactttgaggcagcattcgatgttcctcttaaagtggatctcatcacctttgataccccctttgctggcaggtttggcatcttcacatgctttgatatattgttctttgaccctgccatcagagtcctcagagactacaaggtgaagcatgttgtgtacccaactgcctggatgaaccagctcccactcttggcagcaattgagattcagaaagcttttgctgttgcctttggcatcaacgttctggcagctaatgtccaccacccagttctggggatgacaggaagtggcatacacacccctctggagtccttttggtaccatgacatggaaaatcccaaaagtcaccttataattgcccaggtggccaaaaatccagtgggtctcattggtgcagagaatgcaacaggtgaaacggacccatcccatagtaagtttttaaaaattttgtcaggcgatccgtactgtgagaaggatgctcaggaagtccactgtgatgaggccaccaagtggaacgtgaatgctcctcccacatttcactctgagatgatgtatgacaatttcaccctggtccctgtctggggaaaggaaggctatctccacgtctgttccaatggcctctgctgttatttactttacgagaggcccaccttatccaaagagctgtatgccctgggggtctttgatgggcttcacacagtacatggcacttactacatccaagtgtgtgccctggtcaggtgtgggggtcttggcttcgacacctgtggacaggaaatcacagaggccacggggatatttgagtttcacctgtggggcaacttcagtacttcctatatctttcctttgtttctgacctcagggatgaccctagaagtccctgaccagcttggctgggagaatgaccactatttcctgaggaaaagtaggctgtcctctgggctggtgacggcggctctctatgggcgcttgtatgagagggactaggaaaagtgtgtggtctgtggggcggactctggccatcatgttgacagccttgcacttccacaggctacaagccctgggaccatctttctgccttaagggcaggagcccacttctgtggcaccagattccaccctgggaactgtggaaaaagtaggagaggcagattccctcagtgtcttcctcttaaacctcaatcatcgagacattagggggtattttctgttcacatttatctttttcaagccacatcttcctctaacaaatctctcagtatgcgattggtctcaagctaaaacaaaaataaatgtcagtttatattttacacatccacaaagcagtggcttggggtttttttttttttttttatcttgttgatcaagtgacacccaggacatgtaaatatttcataagccttaaacatttcctgaggtaagaaacaagctctcaaagcaaaagctcaattagaaatggcccttgtggggaaccttcccattctggtcgaccagaactctagccagatgaaatggcaatgctagcgccaccagcaacgtcagaaacgtagaccttaaagcggttttaaaaatagaaaagaagcgttcctcacatctgccagtaatggaattttctgtcagtaaatggaatgtgtaggcaggacctggaataactggagagagtgcaacgcttcggggtgaagggcgggtggggactggaaatgttgagacgggggcagccatgggaaggtatgagtaatagaattctttctgtacgacacagctcatccagggattccaggggaccttaataaatcacggtagctttgggcaagagttgggcacgtcgcccgactgtgcaggatggattgatgctggtattaatttggtctggagccctatagaggatctcgttgctttga

SEQ ID NO: 4 - DNA, 22 bp, Homo sapiens
gccccattacattccagatttg

SEQ ID NO: 5 - DNA, 20 bp, Homo sapiens
gcccaccttatccaaagagc

SEQ ID NO: 6 - DNA, 20 bp, Homo sapiens
gcttggctgggagaatgacc

SEQ ID NO: 7 - DNA, 20 bp, Homo sapiens
ggggaaaggaaggctatctc

SEQ ID NO: 8 - DNA, 20 bp, Homo sapiens
ctccagcgcctgagttgtat

SEQ ID NO: 9 - DNA, 20 bp, Homo sapiens
ctcatacacggcagccacat

SEQ ID NO: 10 - DNA, 19 bp, Homo sapiens
ggtgtcgaagccaagaccc

SEQ ID NO: 11 - DNA, 20 bp, Homo sapiens
cttgtagcctgtggaagtgc

SEQ ID NO: 12 - DNA, 20 bp, Homo sapiens
acaggtgtcgaagccaagac

SEQ ID NO: 13 - DNA, 21 bp, Homo sapiens
tccattattgctgaacacgac

SEQ ID NO: 14 - DNA, 30 bp, Homo sapiens
tggtctgcattatgtctggagccagaagta

SEQ ID NO: 15 - DNA, 30 bp, Homo sapiens
tttgatgggcttcacacagtacatggcact

SEQ ID NO: 16 - DNA, 29 bp, Homo sapiens
agggactaggaaaagtgtgtggtctgtgg

SEQ ID NO: 17 - DNA, 30 bp, Homo sapiens
agggcatacagctctttggataaggtgggc

SEQ ID NO: 18 - DNA, 30 bp, Homo sapiens
aggagccttgtcatagcagtgacccaaggt

SEQ ID NO: 19 - DNA, 20 bp, Homo sapiens
gcttgctcttttcctctgcg

SEQ ID NO: 20 - DNA, 21 bp, Homo sapiens
actacatccacgtgtgtgccc

SEQ ID NO: 21 - DNA, 20 bp, Homo sapiens
ctctatgggcgcttgtatga

SEQ ID NO: 22 - DNA, 20 bp, Homo sapiens
tgaagcccatcaaagacccc

SEQ ID NO: 23 - DNA, 20 bp, Homo sapiens
tggtgaccaatcttgggaca

(19) United States
(12) Patent Application Publication
(10) Pub. No.: US 2004/0219557 A1
(43) Pub. Date: Nov. 4, 2004

(54) Real time PCR assays to detect mutations in the biotinidase gene for newborn screening

(76) Inventors: Steven F. Dobrowolski, Park City, UT (US); Richard A. Banas, Turtle Creek, PA (US); Janine Kennedy, Wexford, PA (US)

Correspondence Address: MCKAY & ASSOCIATES, PC., 801 MCNEILLY ROAD, PITTSBURG, PA, 15226, US

(21) Appl. No.: 10/625,124

(22) Filed: Jul. 23, 2003

Related U.S. Application Data
(60) Provisional application No. 60/400,264, filed on Aug. 1, 2002.

(51) Int. Cl. C12Q 1/68

(52) U.S. Cl. 435/6

(57) ABSTRACT

Biotinidase deficiency is detected by determining the activity of the biotinidase enzyme utilizing a newborn dried blood spot and calorimetric end point analysis. The four mutations most commonly associated with complete biotinidase deficiency are G98: d7i3, Q456H, R538C, and the double mutation D444H:A171T. Partial biotinidase deficiency is almost universally attributed to the D444H mutation. To more effectively distinguish between profound and partial biotinidase deficiency, a panel of assays utilizing real time PCR and melting curve analysis is developed to detect those mutations listed above. In newborn screening for biotinidase deficiency, the analysis of common mutations is useful to distinguish between partial and complete enzyme deficiency. Combining biotinidase enzyme analysis with genotypic data also increases the sensitivity of screening for biotinidase deficiency and provides information useful to clinicians earlier than would otherwise be possible.

### SPECIFIC REFERENCE

The present application claims benefit of provisional application Ser. No. 60/400264, filed Aug. 1, 2002.

### BACKGROUND

1. Description of the Related Art

Biotinidase deficiency is an autosomal recessive metabolic disorder occurring in 1:80,000 live births. Those affected by biotinidase deficiency exhibit irreversible neurological problems, seizures, developmental delays, hypotonia, ataxia, cutaneous and other symptoms. Symptoms are preventable and in some cases reversible through oral biotin supplementation. Prospective newborn screening for biotinidase deficiency is, therefore, performed in much of the USA and in numerous other countries. Biotinidase deficiency results from mutations in the biotinidase gene and depending upon the nature of the mutation(s), the enzyme deficiency may be either complete or partial. Mean biotinidase activity is 7.1 nmol/min/ml serum in normal newborns. Those affected with complete biotinidase deficiency have enzymes that produce <10% of mean normal activity, while those affected with partial deficiency have enzymes that produce 10-30% of normal activity.

In newborn screening laboratories, assaying for biotinidase deficiency is performed using an extract of whole blood derived from the universally collected newborn dried blood spot (DBS). Whole blood extracted from a DBS specimen is an effective sample from which to assay for biotinidase activity, but not as precise as results obtained with serum. Activity of the biotinidase enzyme may be adversely affected if the DBS specimen is mishandled. DBS specimens that are incompletely dried, exposed to moisture after drying, or exposed to heat may exhibit reduced biotinidase activity. Inconclusive or ambiguous results in screening for biotinidase deficiency are therefore often attributable to errors in sample collection and processing prior to their arrival in the screening laboratory. Another difficulty experienced in newborn screening for biotinidase deficiency involves differentiating between a complete and a partial enzyme deficiency. To aid in distinguishing between complete and partial biotinidase deficiency and subsequently increasing the sensitivity and specificity of screening for biotinidase deficiency, mutational analysis has been employed.

In the United States, the following 5 mutations are the most frequently observed in patients with biotinidase deficiency: Q456H, R538C, G98:d7i3, D444H, and the double mutation D444H:A171T. Q456H, R538C, and G98:d7i3 are associated with complete biotinidase deficiency. The D444H mutation has a carrier rate of 3.9% in the general population and causes partial enzyme deficiency. This high frequency in the general population combined with its causing a partial enzyme deficiency makes the D444H mutation similar to the Duarte D2 N314D variant in galactosemia. Interestingly, when D444H is in cis with the A171T mutation, the combined deleterious effects of both mutations result in an allele causing complete enzyme deficiency. The D444H:A171T double mutation is commonly observed in biotinidase deficient children that are ascertained by newborn screening. Using Light Cycler technology and paired hybridization probes, assays were designed to detect these 5 mutations frequently observed in biotinidase deficiency. Reported here are the assay procedures to detect these 5 commonly observed biotinidase mutations together with results from the analysis of specimens identified as presumptively positive in our prospective screening program for biotinidase deficiency. This use of mutational analysis may supplement the biochemical screening results and increases the specificity of newborn screening for biotinidase deficiency.

### BRIEF DESCRIPTION OF THE DRAWINGS

FIG. 1 shows the melting curve results after analysis of the R538C biotinidase mutation.

FIG. 2 shows the melting curve results after analysis of the Q456H biotinidase mutation.

FIG. 3 shows the melting curve results after analysis of the G98:d7i3 biotinidase mutation.

FIG. 4 shows the melting curve results after analysis of the D444H biotinidase mutation.

FIG. 5 shows the melting curve results after analysis of the A171T biotinidase mutation.

### SUMMARY OF THE INVENTION

The present invention relates to newborn screening for biotinidase deficiency using assays involving PCR amplification and Light Cycler platform technologies. 5 common mutations including G98:d7i3, Q456H, R538C, A171T, and D444H are now capable of being detected by a comparison of the hybrid melting temperatures with patient specimens.

Accordingly, what is provided is a method for detecting biotinidase deficiency for newborn screening, comprising amplifying a DNA strand from a specimen to thereby form an amplification product; wherein the amplification product is specific for detecting a mutation frequently observed in patients with biotinidase deficiency; allowing a pair of labeled probes to hybridize to one strand of the amplification product, wherein a detection probe is adapted to match to a sequence that may include the mutation, and an anchor probe hybridizes to an adjacent sequence, thereby forming hybrids; allowing fluorescence resonance energy transfer to occur between a donor fluorophore and an acceptor fluorophore of each hybrid, wherein an excitation wavelength of the donor fluorophore and a fluorescence of the acceptor fluorophore is acquired; and, generating a melting curve having peaks indicative of the melting temperature (Tm) of each hybrid.

### DETAILED DESCRIPTION OF THE PREFERRED EMBODIMENT

Specimens and DNA Preparation

Biotinidase deficient specimens, ascertained through either routine prospective newborn screening or high-risk screening, were retrieved from a specimen archive. Specimens retrieved from archival storage were utilized in assay development and the retrospective study. DNA was isolated from DBS specimens and 80-130 ng was utilized as template in each reaction. Specimens, characterized as homozygous for Q456H, were provided by the Department of Pediatrics, University Hospital Vienna, Vienna, Austria.

Prospective Newborn Screening for Biotinidase Deficiency

DBS specimens are submitted by hospitals. Analysis of biotinidase activity is routinely performed using the Astoria Pacific Continuous Flow Analyzer and the Astoria Pacific SPOTCHECK biotinidase enzyme assay reagents. These reagents, to assay biotinidase activity in DBS specimens, are based upon those methods described by Wolf et al. in a screening method for biotinidase deficiency in newborns, Clin. Chem. (1):125-127, 1984. Samples demonstrating biotinidase activity below the critical cut-off level of 20.0 eru (enzyme response units) are selected for genotype analysis.

Polymerase Chain Reaction and Hybridization Probes

Sequences of the human biotinidase gene (Genbank accession numbers NM00060 (SEQ. ID NO: 1), AF018630 (SEQ. ID NO: 2), AF18631 (SEQ. ID NO: 3) were the basis from which primers and probes were designed. Primer Premier 5 (Premier Biosoft, Palo Alto, Calif.) and Tm Utility 1.5 IT (Idaho Technology, Salt Lake City, Utah) software were utilized as aids to design primers for polymerase chain reaction (PCR) and hybridization probes to detect mutations. Primers and fluorescent labeled probes may also be obtained from either Operon Technology (Alameda, Calif.) or Idaho Technology (Salt Lake City, Utah). PCR reaction buffers may be obtained from Idaho Technology (Salt Lake City, Utah.). Amplification reactions utilize 1× PCR buffer, 2 mM MgCl₂, 200 μM dNTPs (Roche, Manheim, Germany), and 0.6U Klen taq (AB Peptides, St. Louis, Mo.) in a complex with TaqStart antibody (ClonTech, Palo Alto, Calif.). Preparing a complex between the polymerase and TaqStart antibody is performed according to manufacturer's instructions. The sequence of individual primers, the sequence of fluorescent hybridization probes, and the concentration at which each is used are found in Tables 1-3 as follows:

TABLE 1

| | | | | | | |
|---|---|---|---|---|---|---|
| Assay | Forward Primer | conc. | Seq. ID | Reverse Primer | conc. | Seq. ID |

| | | | | | | |
|---|---|---|---|---|---|---|
| G98:d7i3 | GCCCCATTACATTCCAGATTTG | 0.5 | 4 | CTCATACACGGCAGCCACAT | 1.0 | 9 |
| Q456H | GCCCACCTTATCCAAAGAGC | 0.5 | 5 | GGTGTCGAAGCCAAGACCC | 1.0 | 10 |
| R538C | GCTTGGCTGGGAGAATGACC | 0.5 | 6 | CTTGTAGCCTGTGGAAGTGC | 1.0 | 11 |
| D444H | GGGGAAAGGAAGGCTATCTC | 1.0 | 7 | ACAGGTGTCGAAGCCAAGAC | 0.5 | 12 |
| A171T | CTCCAGCGCCTGAGTTGTAT | 0.13 | 8 | TCCATTATTGCTGAACACGAC | 0.25 | 13 |

TABLE 2

| | | |
|---|---|---|
| Assay | Anchor Probe | Seq. ID No. |

| | | |
|---|---|---|
| G98:d7i3 | TGGTCTGCATTATGTCTGGAGCCAGAAGTA-fitc | 14 |
| Q456H | TTTGATGGGCTTCACACAGTACATGGCACT-fitc | 15 |
| R538C | LCred640-AGGGACTAGGAAAAGTGTGTGGTCTGTGG-P | 16 |
| D444H | LCred640-AGGGCATACAGCTCTTTGGATAAGGTGGGC | 17 |
| A171T | LCred640-AGGAGCCTTGTCATAGCAGTGACCCAAGGT-P | 18 |

TABLE 3

| | | |
|---|---|---|
| | | Seq. ID |
| Assay | Detection Probe | No. |

| | | |
|---|---|---|
| G98:d7i3 | LCred640-GCTTGCTCTTTTCCTCTGCG-P | 19 |
| Q456H | LCred640-ACTACATCCACGTGTGTGCCC-P | 20 |
| R538C | CTCTATGGGCGCTTGTATGA-fitc | 21 |
| D444H | TGAAGCCCATCAAAGACCCC-fitc | 22 |
| A17IT | TGGTGACCAATCTTGGGACA-fitc | 23 |

DNA sequences are shown 5′ to 3′, Concentrations are in micromolar units, Anchor probes are utilized at 0.2 micromolar, Detection probes are utilized at 0.1 micromolar, P = phosphate, fitc = fluoroscene isothiocynate, LCred640 = Light Cycler red 640

Ten microliter PCR reactions were performed in capillary tubes using a Roche Light Cycler (Manheim, Germany). Temperature cycling conditions for PCR utilizes a modified 2-step thermal cycling scheme. Specimens are ramped to 94° C. at 20°/second and held there for 0.0 seconds to denature the DNA strands. Temperature then ramps at 20°/second to 58° C. and holds at this temperature for 15 seconds at which time primers anneal and polymerization of new DNA begins. Polymerization is completed while ramping from 58° C. to 72° C. at 1.0°/second. The slow ramp speed allows polymerization to proceed, thus negating the necessity of a hold time at 72° C. Thermal cycling is repeated for 45 cycles. All amplifications are preferably performed in an asymmetric manner. Asymmetric amplifications for G98:d7i3, Q456H, R538C, and A171T assays enrich the antisense strand of the amplicon while the asymmetric amplification in the D444H assay enriches the sense strand of the amplicon. Asymmetry produces an excess of the DNA strand to which the hybridization probes will bind in the analysis phase of the assay.

Hybridization Probes and Genotyping Analysis

Genotyping is performed using paired hybridization probes, where each assay has a detection probe and an anchor probe. Probes for the G98:d7i3, Q456H, R538C, and A171T assays hybridize to the antisense strand of the amplicon while the probes for D444H assay hybridize to the sense strand of the amplicon. The detection probe hybridizes with a region of the amplicon that includes the mutation, while the anchor probe hybridizes with a region adjacent to the detection probe. When both probes are hybridized, there is a 1 base gap between the anchor and detection probes. For each set of hybridization probes, one is conjugated on the 3'end with fitc while the second is conjugated on the 5′ end with LC red640. The probe which is 5′ conjugated with LC red640 is also 3′ phosphorylated to prevent extension by taq DNA polymerase. When both probes are hybridized with the amplicon, the fluorescent moieties are brought into close proximity, and this proximity allows fluorescence resonance energy transfer to occur between the donor fluorophore (fitc) and the acceptor fluorophore (LC red640). Anchor probes have a Tm that is at least 15% higher than the corresponding detection probe, which allows the anchor probe to remain hybridized during the melting transition of the detection probe that occurs during the analysis phase of the assay.

After completing the thermal cycling program, the Light Cycler proceeds seamlessly to the analysis program. The analysis program ramps to 94° C. at 20°/second and after reaching 94° C., immediately begins to ramp at 1°/second to 35° C. Upon reaching 35° C. the temperature ramps upward at 0.1°/second to 76° C. During the entire analysis program, the excitation wavelength of fitc is provided and the fluorescence of LC red640 is continuously acquired. Melting curves are generated by plotting the fluorescence of LC red640 against temperature during the 35°-76° upward temperature ramp. Melting peaks are generated computationally by calculating the −dF/dT of the melting curve which is then plotted against temperature.

### RESULT EXAMPLES

Detecting Frequently Observed Mutations in the Biotinidase Gene.

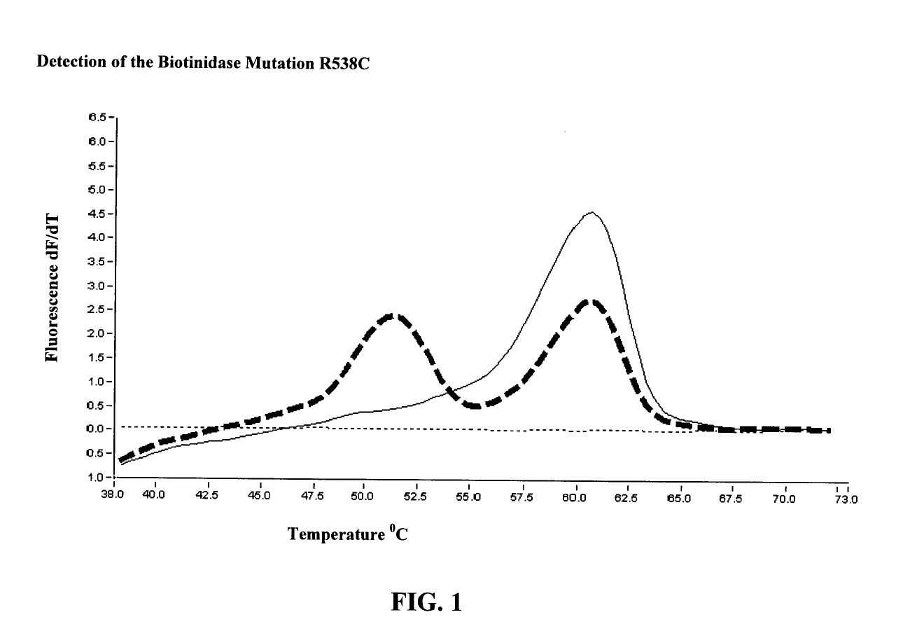

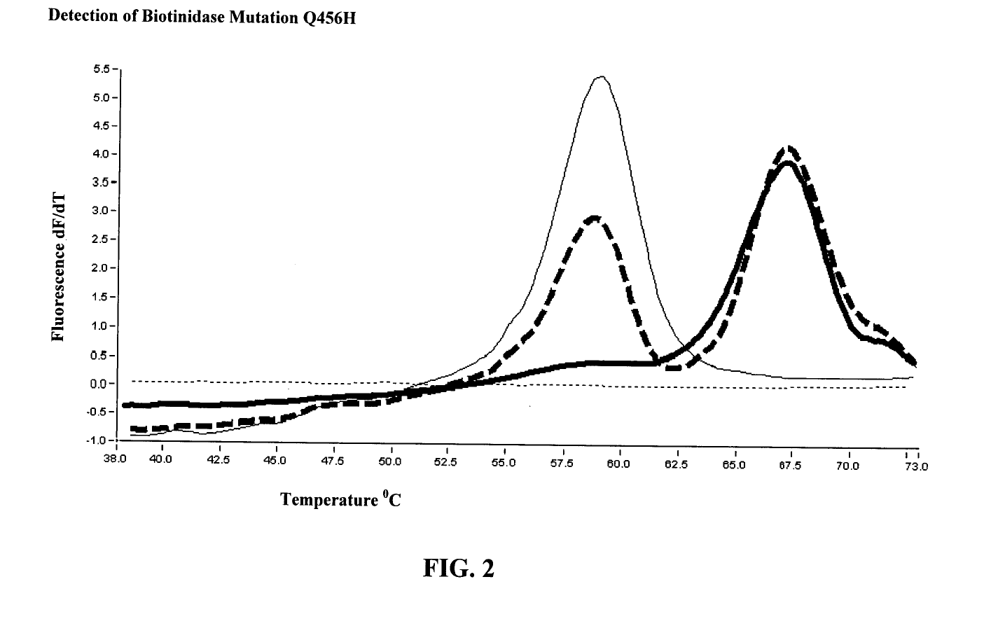

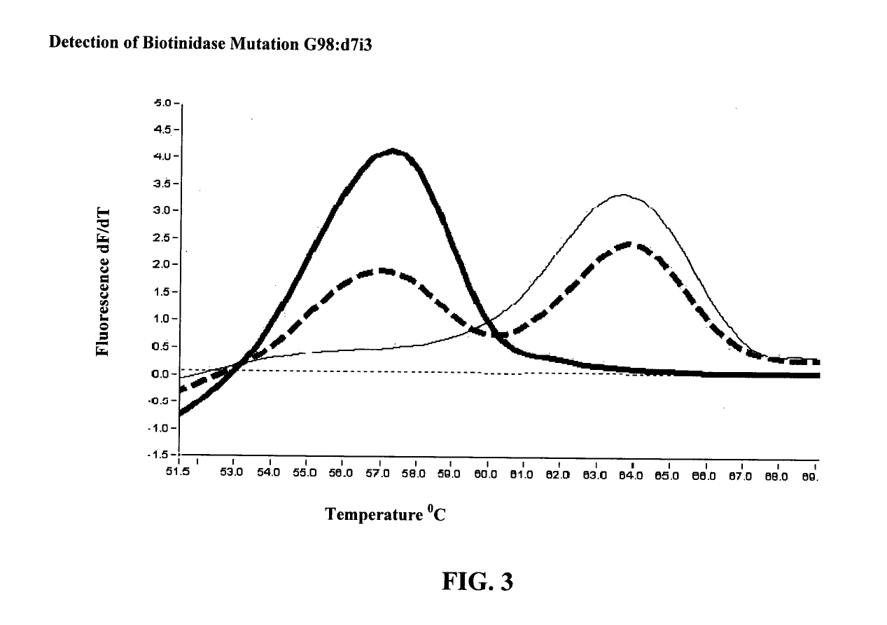

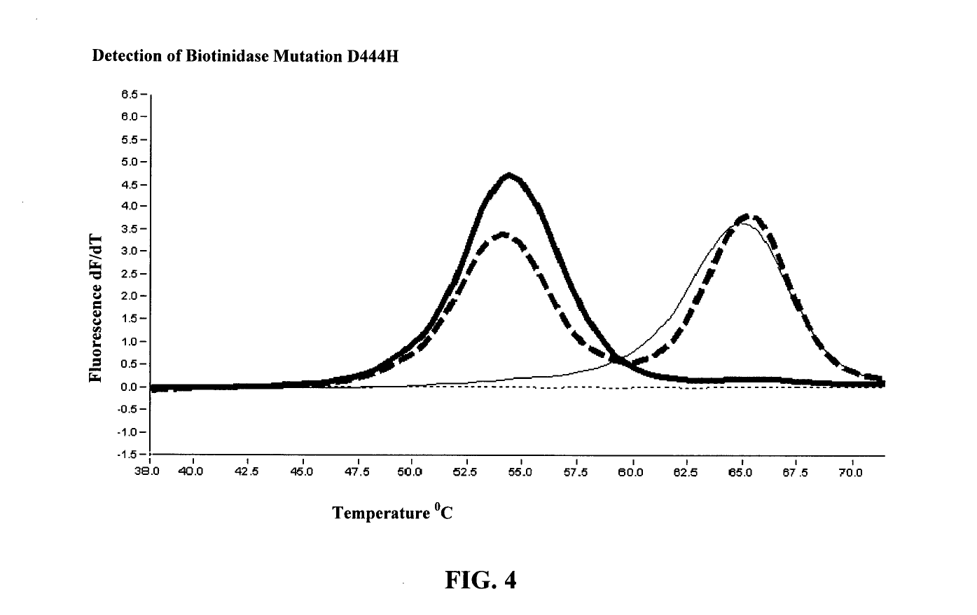

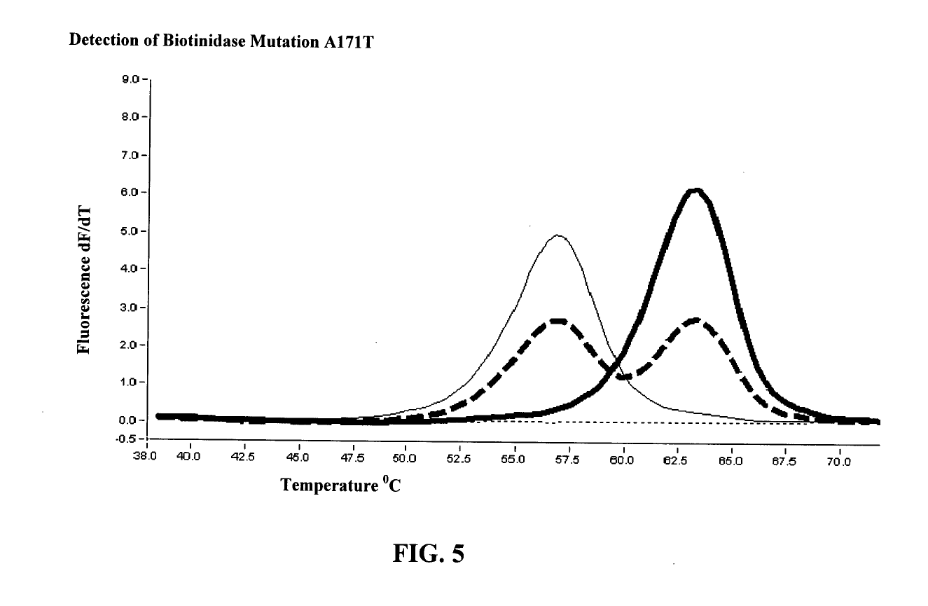

FIGS. 1-5 display analysis of individual biotinidase mutations using melting peaks generated with the Light Cycler. FIG. 1 displays the assay results for the R538C mutation and specimens that are homozygous wild type, heterozygous, and no DNA control are analyzed. No specimen that is homozygous for R538C has yet been identified. The remainder of the assays, shown in FIGS. 2-5 display specimens that are homozygous wild type, heterozygous, homozygous for the mutation, and a no DNA control. In the cases of the D444H, G98:d7i3, and R538C assays, the detection probe matches the wild type sequence. Therefore, the high temperature melting peak represents the wild type form of the gene while the low temperature melting peak represents the mutant form of the gene. In the A171T and Q456H assays, the detection probe matches the mutant form of the gene and has a 1 base pair mismatch with the wild type allele. In these assays, the high-temperature melting peak represents the mutant form of the gene while the low-temperature melting peak represents the wild type form of the gene.

Analysis of Specimens Identified through Newborn Screening as Presumptive Positive for Biotinidase Deficiency.

Through newborn screening, 49 specimens were identified as presumptively positive for biotinidase deficiency. Of these 49 specimens, 45 were suitable for genotype analysis. In the cases of the 4 specimens that were not analyzed, there was inadequate dried blood remaining on the DBS to obtain a DNA specimen. These 45 specimens were analyzed for the 5 mutations and genotyping results are shown in Table 4 as follows:

TABLE 4

| | | | | Genotype |
|---|---|---|---|---|
| Spec- | | | | Based Preliminary |
| imen | Origin | Allele 1 | Allele 2 | Diagnosis |
| BD1 | Domestic | D444H/A171T | D444H | Partial Deficiency |
| BD2 | Domestic | Q456H | ND | Incomplete |
| | | | | Genotype |
| BD3 | Domestic | G98:d7i3 | D444H | Partial Deficiency |
| BD4 | Domestic | Q456H | ND | Incomplete |
| | | | | Genotype |
| BD5 | Domestic | ND | ND | — |
| BD6 | Domestic | G98:d7i3 | ND | Incomplete |
| | | | | Genotype |
| BD7 | Domestic | Q456H | ND | Incomplete |
| | | | | Genotype |
| BD8 | Domestic | Q456H | ND | Incomplete |
| | | | | Genotype |
| BD9 | Brazil | ND | ND | — |
| BD10 | Domestic | D444H/AI7IT | R538C | Complete |
| | | | | Deficiency |
| BD11 | Turkey | D444H | D444H | Partial Deficiency |
| BD12 | Domestic | Q456H | D444H | Partial Deficiency |
| BD13 | Chile | D444H | ND | Putative |
| | | | | Partial Deficiency |
| BD14 | India | G98:d7i3 | G98:d7i3 | Complete |
| | | | | Deficiency |
| BD15 | Mexico | D444H | ND | Putative |
| | | | | Partial Deficiency |
| BD16 | Chile | ND | ND | — |
| BD17 | Domestic | ND | ND | — |
| BD18 | Domestic | ND | ND | — |
| BD19 | Domestic | D444H | D444H | Partial Deficiency |
| BD20 | Domestic | Q456H | ND | Incomplete |
| | | | | Genotype |
| BD21 | Domestic | D444H | ND | Putative |
| | | | | Partial Deficiency |
| BD22 | Brazil | G98:d7i3 | ND | Incomplete |
| | | | | Genotype |
| BD23 | India | G98:d7i3 | G98:d7i3 | Complete |
| | | | | Deficiency |
| BD24 | Domestic | D444/A171T | D444H/A171T | Complete |
| | | | | Deficiency |
| BD25 | Domestic | D444H | ND | Putative |
| | | | | Partial Deficiency |
| BD26 | Domestic | Q456H | D444H | Partial Deficiency |
| BD27 | Domestic | D444H/A171T | D444H | Partial Deficiency |
| BD28 | Domestic | D444H | D444H | Partial Deficiency |
| BD29 | Domestic | D444H | D444H | Partial Deficiency |
| BD30 | Domestic | D444H | D444H | Partial Deficiency |
| BD31 | Domestic | D444H | D444H | Partial Deficiency |
| BD32 | Domestic | D444H | ND | Putative |
| | | | | Partial Deficiency |
| BD33 | Brazil | D444H | D444H | Partial Deficiency |
| BD34 | Domestic | Q4565H | ND | Incomplete |
| | | | | Genotype |
| BD35 | Domestic | R538C | D444H | Partial Deficiency |
| BD36 | Domestic | D444H | ND | Putative |
| | | | | Partial Deficiency |
| BD37 | Domestic | Q456H | Q456H | Complete |
| | | | | Deficiency |
| BD38 | Domestic | D444H:A171T | ND | Incomplete |
| | | | | Genotype |
| BD39 | Domestic | Q456H | D444H | Partial Deficiency |
| BD40 | Domestic | D444H | ND | Putative |
| | | | | Partial Deficiency |
| BD41 | Domestic | Q456H | ND | Incomplete |
| | | | | Genotype |
| BD42 | Domestic | D444H | ND | Putative |
| | | | | Partial Deficiency |
| BD43 | Domestic | Q456H | D444H | Partial Deficiency |
| BD44 | Domestic | R538C | D444H | Partial Deficiency |
| BD45 | Domestic | D444H | D444H | Partial Deficiency |

ND no mutation detected.

Thirty-six specimens were of domestic origin and 9 were of foreign origin (see table 4 for the countries of origin). Overall, in 88.8% (40/45) of the specimens at least 1 mutation was identified. For specimens of domestic origin, 91.6% (33/36) contained at least 1 mutation, while 78% of specimens of foreign origin (7/9) contained at least 1 mutation. A complete genotype was obtained from 21 specimens. Seventeen specimens (BD1, BD3, BD11, BD12, BD19, BD26-31, BD33, BD35, BD39, BD43-45) could be assigned a preliminary designation of partial deficiency because they were either homozygous for D444H or were compound heterozygous between D444H and one of the other mutations being assayed for. The genotypes of 5 specimens (BD10, BD14, BD23, BD24, BD37) clearly identified them as complete deficiencies, 2 of which were homozygous for G98:d7i3 (BD14, BD23), one was homozygous for D444H:A171T (BD24), one was homozygous for Q456H (BD37), and one was a compound heterozygote for D444H:A171T and R538C (BD10). Eight additional specimens (BD13, BD15, BD21, BD25, BD32, BD36, BD40, BD42) could be assigned a preliminary designation of partial deficiency owing to the presence of a single copy of the D444H mutation and reduced enzyme activity. Mutations observed in specimens of foreign origin were limited to D444H and G98:d7i3, while all 5 mutations were observed in specimens of domestic origin.

In newborn screening for biotinidase deficiency, it is frequently difficult to discern if a partial or complete enzyme deficiency has been encountered. In the vast majority of partial deficiencies, the D444H mutation is involved (8). D444H has a carrier frequency of 3.9% in the general United States population and reduces the activity of the biotinidase enzyme by 48-52%. It is noteworthy, that these percent reductions were determined with serum quantitative enzyme analysis, thus the percent enzyme reduction in a DBS derived whole blood specimen could be greater. Partial deficiencies are either homozygous for D444H or compound heterozygotes with D444H and a second mutation. In Table 2, there are seventeen specimens with genotypes identifying them as partial deficiencies. Nine specimens are compound heterozygous with D444H and a second mutation, while eight are homozygous for D444H. Additionally, there are eight other specimens where a single copy of D444H is identified. This is strong evidence that these too are partial deficiencies. It is unlikely that these eight specimens with a single copy of D444H are simple carriers because the enzyme assay was below the critical cut-off and a carrier of D444H would not be expected to produce such low enzyme activity. This suggests that such specimens are likely compound heterozygotes having one copy of D444H and a rare or private mutation in the second copy of the biotinidase gene. In the enzyme assay used in newborn screening, compound heterozygotes containing D444H and a mutation causing a complete deficiency (R538C, G98:d7i3, Q456H, etc) may generate biochemical data effectively mimicking complete biotinidase deficiency. A similar situation is frequently observed in the Beutler assay that is used to measure GALT activity when screening for galactosemia. Compound heterozygotes between the Duarte D2 N314D variant and a classical galactosemia mutation such as Q188R may generate biochemical data suggesting classical galactosemia. Identifying the N314D GALT mutation provides definitive proof that these specimens are not classical galactosemia. In a similar fashion, the D444H mutation is responsible for the vast majority of partial biotinidase deficiencies and therefore identifying this mutation provides strong evidence that a complete enzyme deficiency is not present.

A complication surrounding the D444H mutation is double mutants. Three double mutations, involving D444H and a second mutation on the same gene, have been described. The commonly observed double mutant, D444H:A171T, that accounts for 17.3% of the mutations in complete biotinidase deficiencies ascertained by newborn screening, is part of this panel. Indeed, specimen BD24 from Table 2 is homozygous for D444H:A171T. Two other double mutations, D444H:F403V and D444H:R157H, have been described, however both are extremely rare. The D444H mutation is very useful to identify partial deficiencies, but the possibility of a rare or unique double mutant resulting in a complete deficiency cannot be dismissed. After newborn screening results are reported, the first clinical visit of a potential biotinidase deficient newborn will involve determining quantitative serum biotinidase activity and possibly confirmatory molecular diagnostic analysis. Quantitative biotinidase analysis is the ultimate diagnostic test to identify biotinidase deficiency and the newborn screening analysis is secondary to these results. Second tier mutation screening is to benefit the newborn screening program and acts as a guide in clinical evaluation. However in certain situations, as are observed in specimens BD10, BD14, BD23, BD24, and BD37, the genotype data unambiguously identifies these specimens as having a complete enzyme deficiency. Such informative results can expedite patient care to get the newborn immediate attention.

The data shown in Table 4 and discussed above provides evidence to the utility of second tier mutation analysis in newborn screening for biotinidase deficiency. In a high throughput newborn screening laboratory, the most important issue is validity of results, but following closely behind is turn around time. Minimizing turn around time requires that assay platforms be fast, reliable. and easily interpreted within the context of a routine service laboratory. The Light Cycler platform is ideal for the high throughput newborn screening laboratory because all of these criteria are met. Air driven thermal cycling is fast, genotyping with fluorescent hybridization probes is simple because it involves no post-PCR manipulation, and melting peak data is easily interpreted. From isolation of DNA to data interpretation, the 5-mutation panel described here is completed in less than 2 hours. Such rapid analysis assures that second tier molecular data is reported along with the primary biochemical data. An additional benefit is that the close tube format simplifies sample tracking and is favorable for avoiding amplicon contamination in the laboratory. Data files from the Light Cycler are easily stored and may be backed up in an off-site archive rendering them safe from loss. This is an ideal situation for the newborn screening laboratory where large quantities of sensitive clinical data are generated.

In the example above, mutations that cause biotinidase deficiency were identified. Among specimens of domestic origin identified using the present methodology, the panel of five mutations proved useful in 91.6 % of presumptive positive newborns. Biochemical analysis will remain the primary means by which biotinidase deficiency is detected in both newborn screening and clinical diagnostics. Second tier mutation analysis provides valuable support to biochemical analysis and should be considered as a supplement to the biochemical data by those performing newborn screening for biotinidase deficiency.

I claim:

1. A method for detecting biotinidase deficiency for newborn screening, comprising:
amplifying a DNA strand from a specimen to thereby form an amplification product; wherein said amplification product is specific for detecting a mutation frequently observed in patients with said biotinidase deficiency;
allowing a pair of labeled probes to hybridize to one strand of said amplification product, wherein a detection probe is adapted to match to a sequence that may include said mutation, and an anchor probe hybridizes to an adjacent sequence, thereby forming hybrids;
allowing fluorescence resonance energy transfer to occur between a donor fluorophore and an acceptor fluorophore of each said hybrid, wherein an excitation wavelength of said donor fluorophore and a fluorescence of said acceptor fluorophore is acquired; and,
generating a melting curve having peaks indicative of the melting temperature (Tm) of each said hybrid.

2. The method of claim 1, wherein said mutations are selected from the group consisting of G98:d7i3, Q456H, R538C, D444H, and A171T.

3. The method of claim 1, wherein for the step of amplifying said DNA strand, such amplification is performed in an asymmetric manner.

4. The method of claim 1, wherein for the step of amplifying said DNA strand, a forward primer selected from the group consisting of those such sequences as set forth in SEQ ID NO: 4, SEQ ID NO: 5, SEQ ID NO: 6, SEQ ID NO 7, and SEQ ID NO: 8 is used.

5. The method of claim 1, wherein for the step of amplifying said DNA strand, a reverse primer selected from the group consisting of those such sequences as set forth in SEQ ID NO: 9, SEQ ID NO: 10, SEQ ID NO: 11, SEQ ID NO 12, and SEQ ID NO: 13 is used.

6. The method of claim 1, wherein said detection probe is selected from the group consisting of those such sequences as set forth in SEQ ID NO: 19, SEQ ID NO: 20, SEQ ID NO 21, SEQ ID NO: 22, and SEQ ID NO: 23.

7. The method of claim 6, wherein said detection probe is conjugated with LC red640.

8. The method of claim 7, wherein said detection probe is phosphorylated.

9. The method of claim 6, wherein said detection probe is conjugated with fitc.

10. The method of claim 1, wherein said anchor probe is selected from the group consisting of those such sequences as set forth in SEQ ID NO: 14, SEQ ID NO: 15, SEQ ID NO 16, SEQ ID NO: 17, and SEQ ID NO: 18.

11. The method of claim 10, wherein said anchor probe is conjugated with LC red640.

12. The method of claim 11, wherein said anchor probe is phosphorylated.

13. The method of claim 10, wherein said anchor probe is conjugated with fitc.

14. The method of claim 1, wherein for the step of generating said melting curves, said fluorescence of said acceptor fluorophore is plotted against a temperature during a 35°-76° upward temperature ramp.

15. A method for detecting biotinidase deficiency for newborn screening, comprising:
amplifying a DNA strand from a specimen to thereby form an amplification product;
allowing a pair of labeled probes to hybridize to one strand of said amplification product, wherein one of said labeled probes is a detection probe selected from the group consisting of those such sequences as set forth in SEQ ID NO: 19, SEQ ID NO: 20, SEQ ID NO 21, SEQ ID NO: 22, and SEQ ID NO: 23, and wherein one of said labeled probes is an anchor probe selected from the group consisting of those such sequences as set forth in SEQ ID NO: 14, SEQ ID NO: 15, SEQ ID NO 16, SEQ ID NO: 17, and SEQ ID NO: 18; thereby forming hybrids;
allowing fluorescence resonance energy transfer to occur between a donor fluorophore and an acceptor fluorophore of each said hybrid, wherein an excitation wavelength of said donor fluorophore and a fluorescence of said acceptor fluorophore is acquired; and,
generating a melting curve having peaks indicative of the melting temperature (Tm) of each said hybrid.

16. The method of claim 15, wherein for the step of amplifying said DNA strand, such amplification is performed in an asymmetric manner.

17. The method of claim 15, wherein for the step of amplifying said DNA strand, a forward primer selected from the group consisting of those such sequences as set forth in SEQ ID NO: 4, SEQ ID NO: 5, SEQ ID NO: 6, SEQ ID NO 7, and SEQ ID NO: 8 is used.

18. The method of claim 15, wherein for the step of amplifying said DNA strand, a reverse primer selected from the group consisting of those such sequences as set forth in SEQ ID NO: 9, SEQ ID NO: 10, SEQ ID NO: 11, SEQ ID NO 12, and SEQ ID NO: 13 is used.

19. The method of claim 15, wherein said detection probe is conjugated with LC red640.

20. The method of claim 19, wherein said detection probe is phosphorylated.

21. The method of claim 15, wherein said detection probe is conjugated with fitc.

22. The method of claim 15, wherein said anchor probe is conjugated with LC red640.

23. The method of claim 22, wherein said anchor probe is phosphorylated.

24. The method of claim 15, wherein said anchor probe is conjugated with fitc.
